(19) United States
(12) Patent Application Publication
(10) Pub. No.: US 2004/0029805 A1
(43) Pub. Date: Feb. 12, 2004

(54) Prevention and treatment of nonalcoholic fatty liver disease (NAFLD) by antagonism of the receptor to glucose-dependent insulinotropic polypeptide (GIP)

(76) Inventors: M. Michael Wolfe, Newton, MA (US); Michael O. Boylan, Milton, MA (US)

Correspondence Address: EDWARDS & ANGELL, LLP, P.O. BOX 9169, BOSTON, MA, 02209, US

(21) Appl. No.: 10/461,655

(22) Filed: Jun. 13, 2003

Related U.S. Application Data
(60) Provisional application No. 60/389,320, filed on Jun. 15, 2002.

(51) Int. Cl. A61K 38/17; A61K 48/00

(52) U.S. Cl. 514/12; 514/44

(57) ABSTRACT

The present invention concerns the use of various forms of GIP-receptor antagonists to attenuate the insulin response to GIP following meals in animals, such as humans, to prevent, reduce, inhibit and/or treat nonalcoholic fatty liver disease by virtue of its prevention and/or reversal of hyperinsulinemia and insulin resistance. Thus, the use of the GIP-receptor antagonists in any effective form is believed to prevent the development and reverse the process of NAFLD. The present invention is accomplished by administering an effective amount of an antagonistic agent, such as a GIP antagonist or an antisense molecule, to antagonize, block, inhibit or ablate the receptor to Glucose-Dependent Insulinotropic Polypeptide (GIP).

### RELATED U.S. PATENT APPLICATION

This application for U.S. Patent relates and claims priority to U.S. provisional application Serial No. 60/389,320, filed Jun. 15, 2002, entitled Prevention and Treatment of Nonalcoholic Fatty Liver Disease (NAFLD) by Antagonism of the Receptor to Glucose-Dependent Insulinotropic polypeptide(GIP), and is incorporated herein by reference in its entirety.

### FIELD OF THE INVENTION

Nonalcoholic fatty liver disease (NAFLD) is a disorder with histologic features of alcohol-induced liver disease that occurs in people who do not consume significant amounts of alcohol. Several studies have suggested that this entity is uncommon and that it occurs most often in middle-aged, overweight females. Hyperglycemia with and without evidence of hyperlipidemia is commonly associated with NAFLD and is felt to be a predisposing condition. More recent reports have suggested that NAFLD may be more common than originally suspected and that it may affect individuals who lack the typical risk factors for this disorder. At the present time, the clinical implications of NAFLD have not been clearly defined, although progression to cirrhosis has been noted in many cases. Unfortunately, prior efforts to prevent or treat NAFLD have been limited by a poor understanding of the pathogenesis of this disorder.

### BACKGROUND

Recent studies have speculated that NAFLD may represent the end result of several diverse insults. These reports imply that the pathogenesis of NAFLD may be multifactorial and the mechanisms underlying this entity include an amino acid imbalance and endotoxemia associated with overwhelming infection or starvation-associated bacterial translocation. Most recently, studies have suggested that this entity may be due most commonly to hyperinsulinemia and insulin resistance.

### SUMMARY

Glucose-dependent insulinotropic polypeptide (GIP) is a hormone secreted by K-cells of the upper small intestine. Although GIP was originally regarded as an inhibitor of gastric acid secretion, more recent studies suggest that its principal physiological property may be its inherent ability to stimulate the release of insulin from pancreatic $-islet cells of the pancreas. GIP is thus felt to play an important physiological role in maintaining glucose balance following meals containing not only glucose, but also fat.

In U.S. patent applications, Ser. No. 10/003,674, filed on Oct. 23, 2001 and entitled “Specific Antagonists for Glucose-Dependent Insulinotropic Polypeptide (GIP)”, which is a continuation of U.S. patent application, Ser. No. 08/984,476, filed on Dec. 3, 1997, which claims priority from U.S. provisional application, Serial No. 60/032,329, filed Dec. 12, 1996, which are incorporated herein by reference in their entireties, disclose examples of receptor antagonists to GIP that have been shown to successfully inhibit the release of insulin in laboratory animals. The present invention involves the use of these GIP-specific receptor antagonists to prevent, treat and/or decrease hyperinsulinemia and thereby prevent both insulin resistance and the development of nonalcoholic fatty liver disease.

Thus, the present invention proposes the use of various forms of the GIP-receptor antagonist to attenuate the insulin response to GIP following meals in animals, such as humans. It is proposed that the use of the GIP-receptor antagonists in any form will thus prevent the development and reverse the process of NAFLD. The GIP receptor can be antagonized using several different methods, including a peptide antagonist, which is identical or similar to 7-30 GIP(NH₂) or any similar peptide that effectively antagonizes the GIP-receptor, such as those set forth in Table I. In addition to peptide antagonists, the possibility of using a nonpeptide receptor antagonist is contemplated by the present invention as is the use of antisense recombinant technology or any other method which effectively antagonizes the GIP Receptor.

Insert sequences.

TABLE 1

```
            1
            30
            PRT
            Homo sapiens
            1
Tyr Ala Glu Gly Thr Phe Ile Ser Asp Tyr Ser Ile Ala Met Asp Lys
  1 5 10 15
Ile His Gln Gln Asp Phe Val Asn Trp Leu Leu Ala Gln Lys
                        20 25 30
            2
            24
            PRT
            Homo sapiens
            2
Ile Ser Asp Tyr Ser Ile Ala Met Asp Lys Ile His Gln Gln Asp Ph e
  1 5 10 15
Val Asn Trp Leu Leu Ala Gln Lys
                        20
            3
            15
            PRT
            Hono sapiens
            3
Lys Ile His Gln Gln Asp Phe Val Asn Trp Leu Leu Ala Gln Lys
  1 5 10 15
            4
            5
            PRT
            Home sapiens or Rattus nervegicus
            4
Ile Ser Asp Tyr Ser Ile Ala Met Asp
  1 5
            5
            21
            PRT
            Homo sapiens
            5
Tyr Ser Ile Ala Met Asp Lys Ile His Gln Gln Asp Phe Val Asn Trp
  1 5 10 15
Leu Leu Ala Gln Lys
                        20
            6
            3
            PRT
            Homo sapeins or Rattus nervegicus
            6
Ile Ser Asp
  1
            7
            30
            PRT
            Rattus nervegicus
            7
Tyr Ala Glu Gly Thr Phe Ile Ser Asp Tyr Ser Ile Ala Met Asp Lys
  1 5 10 15
Ile Arg Gln Gln Asp Phe Val Asn Trp Leu Leu Ala Gln Lys
                        20 25 30
            8
            24
            PRT
            Rattus nervegicus
            8
Ile Ser Asp Tyr Ser Ile Ala Met Asp Lys Ile Arg Gln Gln Asp Phe
  1 5 10 15
Val Asn Trp Leu Leu Ala Gln Lys
                        20
            9
            15
            Rattus nervegicus
            9
Lys Ile Arg Gln Gln Asp Phe Val Asn Trp Leu Leu Ala Gln Lys
  1 5 10 15
            10
            21
            PRT
            Rattus nervegicus
            10
Tyr Ser Ile Ala Met Asp Lys Ile Arg Gln Gln Asp Phe Val Asn Trp
  1 5 10 15
Leu Leu Ala Gln Lys
                        20
            11
            22
            PRT
            Homo sapiens
            11
Tyr Ala Glu Gly Thr Phe Ile Ser Asp Tyr Ser Ile Ala Met Asp Lys
  1 5 10 15
Ile His Gln Gln Asp Phe Val Asn Trp Leu Leu Ala Gln Lys Gly Lys
                        20 25 30
Lys Asn Asp Trp Lys His Asn Ile Thr Gln
                35 40
            12
            42
            PRT
            Rattus nervegicus
            12
Tyr Ala Glu Gly Thr Phe Ile Ser Asp Tyr Ser Ile Ala Met Asp Lys
  1 5 10 15
Ile Arg Gln Gln Asp Phe Val Asn Trp Leu Leu Ala Gln Lys Gly Lys
                        20 25 30
Lys Asn Asp Trp Lys His Asn Ile Thr Gln
                35 40
            13
            10
            PRT
            Homo sapiens or Rattus nervegicus
            13
Asp Phe Val Asn Trp Leu Leu Ala Gln Lys
  1 5 10
            14
            14
            PRT
            Rattus nervegicus
            14
Gly Lys Lys Asn Asp Trp Lys His Asn Leu Thr Gln Arg Glu
  1 5 10
```

An example of the potential use of GIP receptor antagonist in accordance with the present invention is in connection with a forty-five year old woman with no significant past medical history with abnormal liver enzymes. The patient denies any significant use of alcohol, and various serological tests for hepatitis-associated viruses are negative. These viruses include Hepatitis A, B, and C, as well as Epstein-Barr virus and cytomegalovirus. Moreover, serology for the possibility for autoimmune liver disease, including ANA, ASMA, AMA, and LKM microsomal antibodies, are all negative. Finally, a metabolic profile testing for iron overload, Wilson's Disease, and “−1 antitrypsin deficiency are all negative. An ultrasound of patient's liver and gallbladder reveal a fatty liver and a biopsy with the liver is consistent with NAFLD. Although the patient is non-obese, she is found to be hyperinsulinemic. Because no specific therapy is presently available for this condition, only supportive measures can be recommended to the patient's primary care physician.

Consistent with the present invention, a patient that presents symptoms as described in this Example may be treated with an effective amount of a GIP receptor antagonist.

As indicated herein, this may be accomplished by injection, oral administration or gene manipulation (i.e., gene therapy). When administered orally, a GIP receptor antagonist may be given, for example, from 1 to about 6 times daily. However, if the GIP receptor antagonist is administered by injection, it may be given, for example, from about once per month to about four or more times per day. When gene therapy is chosen as the route of administration, an effective amount of a GIP receptor antagonist may be delivered from once per lifetime to about once per month or more.

As indicated herein above, GIP receptor antagonists in accordance with the present invention may be in the form of a peptide or nonpeptide antagonist, a small chemical entity, antisense DNA sequence or any other form which can effectively accomplish the objectives of the present invention.

It is currently believed that an effective amount of a GIP receptor antagonist is an amount that is sufficient to inhibit GIP or GIP receptor activity by approximately 10% to about 100% and more preferably by approximately 40% to about 100% and more preferably by approximately 50% to about 100% or by approximately 40% to about 80% or by approximately 50% to about 80% or by approximately 40% to about 75% or by approximately 50% to about 75% and/or insulin by approximately 10% to about 100% and more preferably by approximately 40% to about 100% and more preferably by approximately 50% to about 100% or by approximately 40% to about 80% or by approximately 50% to about 80% or by approximately 40% to about 75% or by approximately 50% to about 75%.

Thus, the present invention concerns the use of an antagonist to the GIP-receptor to prevent, reduce, inhibit and/or treat nonalcoholic fatty liver disease by virtue of its prevention and/or reversal of hyperinsulinemia and insulin resistance.

Therapeutic compositions according to this invention are formulated in pharmaceutical compositions containing one or more antagonistic agents, e.g., GIP antagonists, and a pharmaceutically acceptable carrier. The pharmaceutical compositions in accordance with the present invention may contain other components so long as the other components do not reduce or interfere with the effectiveness of the agent antagonists according to the objectives of this invention so much that therapy is negated or limited. Examples of such compositions include sweetening, flavoring, coloring, dispersing, disintegrating, binding, granulating, suspending, wetting, preservative and demulcent agents and the like. Pharmaceutically acceptable carriers are well known, and one skilled in the pharmaceutical art can easily select carriers suitable for particular-routes of administration.

Also contemplated by the present invention, the antagonist agents, such as the GIP antagonists, may be lyophilized using standard techniques known to those skilled in the lyophilized art. The lyophilized antagonistic agent may be reconstituted at the time of use with, for example, suitable diluents such as normal saline, sterile water, glacial acetic acid, sodium acetate, combinations thereof and the like. Once reconstituted, the antagonistic agents may be administered parentally or orally and may further include preservatives and/or other acceptable inert components as mentioned hereinbefore.

The pharmaceutical compositions containing any of the antagonistic agents, e.g., GIP antagonists, in accordance with the present invention may be administered by parenteral (subcutaneously, intramuscularly, intravenously, intraperitoneally, intrapleurally, or intravesicularly or intrathecally), gene therapy, topical, oral, rectal or nasal route, as necessitated by the choice of drug and disease. The dose used in a particular formulation or application will be determined by the requirements of the particular state of the disease, type of treatment, and the constraints imposed by the capacities of the carrier materials. The concentrations of the active ingredient in pharmaceutically acceptable carriers may range from about 0.1 nM to about 100 μM or more. The compositions described herein above may be combined or used together in coordination with other therapeutic substances so long as the objectives of the present invention are not defeated.

Dose will depend upon a variety of factors, including the therapeutic index of the drugs, disease type, patient age, patient weight, and tolerance activity. Doses will generally be chosen to achieve serum concentrations from about 0.1 nM to about 100 M or more. Preferably, initial dose levels will be selected based upon their ability to achieve ambient concentrations shown to be effective in vivo models, such as that used to determine therapeutic index, and in vivo models and in clinical trials, up to maximum tolerated or treatment-limiting levels.

Accordingly, it will be understood that embodiments of the present invention have been disclosed by way of example and that other modifications and alterations may occur to those skilled in the art without departing from the scope and spirit of the appended claims. Thus, the invention described herein extends to all such modifications and variations as will be apparent to the reader skilled in the art, and also extends to combinations and sub-combinations of the features of this description, including those described in U.S. patent applications, Ser. No. 10/003,674, filed on Oct. 23, 2001 and entitled “Specific Antagonists for Glucose-Dependent Insulinotropic Polypeptide (GIP)”, which is a continuation of U.S. patent application, Ser. No. 08/984,476, filed on Dec. 3, 1997, which claims priority from U.S. provisional application, Serial No. 60/032,329, filed Dec. 12, 1996, which are incorporated herein by reference in their entireties.

It will also be understood that, although preferred embodiments of the present invention have been illustrated in Table I, are set forth in U.S. patent application, Ser. No. 10/003,674, Ser. No. 08/984,476 and Ser. No. 60/032,329, and described in the foregoing detailed description and example, the invention is not limited to the embodiments disclosed, but is capable of numerous rearrangements, modifications and substitutions without departing from the spirit of the invention as set forth and defined by the following claims.

We claim:

1. A method of preventing, inhibiting, treating or reducing nonalcoholic fatty liver disease in an animal comprising:
administering an effective amount of an agent to antagonize, block, inhibit or ablate the receptor to Glucose-Dependent Insulinotropic Polypeptide (GIP) to prevent, inhibit, treat or reduce nonalcoholic fatty liver disease in the animal.

2. A method of claim 1, wherein the agent is a GIP receptor antagonist.

3. A method of claim 2, wherein the GIP receptor antagonist is selected from the group consisting of those set forth in Table I.

4. A method of claim 1 wherein the agent is administered to the animal orally, by injection or by gene therapy.

5. A method of claim 2, wherein the GIP receptor antagonist comprises a 24 amino polypeptide corresponding to positions 7-30 of the sequence of GIP or effective alternatives thereto.

6. A method of claim 2, wherein the GIP receptor antagonist comprises at least an effective number of amino acids corresponding to positions 7-30 of the sequence of GIP or effective alternatives thereto.

7. A method of claim 1, wherein the agent is administered as a pharmaceutical comprising the agent and an acceptable pharmaceutical carrier.

8. A method of claim 7, wherein the agent is a GIP receptor antagonist.

9. A method of claim 8, wherein the GIP receptor antagonist comprises a 24 amino polypeptide corresponding to positions 7-30 of the sequence of GIP or effective alternatives thereto.

10. A method of claim 8, wherein the GIP receptor antagonist comprises at least an effective number of amino acids corresponding to positions 7-30 of the sequence of GIP or effective alternatives thereto.

11. A method of claim 7, wherein the pharmaceutical composition further includes an inert pharmaceutical excipient selected from the group consisting of sweetening, flavoring, coloring, dispersing, disintegrating, binding, granulating, suspending, wetting, preservative and demulcent excipients.

12. A method of claims 7, 8, 9 or 10, wherein the agent is lyophilized.

13. A method of claim 12, wherein the lyophilized agent is reconstituted with a suitable diluent selected from the group consisting of normal saline, sterile water, glacial acetic acid, sodium acetate and combinations thereof.

14. A method of preventing, inhibiting, treating or reducing nonalcoholic fatty liver disease in an animal comprising:
administering an effective amount of an agent to prevent and/or reverse hyperinsulinemia and/or insulin resistance to prevent, inhibit, treat or reduce nonalcoholic fatty liver disease in the animal.

15. A method of claim 2, wherein the agent is a GIP receptor antagonist.

16. A method of claim 3, wherein the GIP receptor antagonist is selected from the group consisting of those set forth in Table I.

17. A method of claim 1 wherein the agent is administered to the animal orally, by injection or by gene therapy.

18. A method of claim 15, wherein the GIP receptor antagonist comprises a 24 amino polypeptide corresponding to positions 7-30 of the sequence of GIP or effective alternatives thereto.

19. A method of claim 15, wherein the GIP receptor antagonist comprises at least an effective number of amino acids corresponding to positions 7-30 of the sequence of GIP or effective alternatives thereto.

20. A method of claim 14, wherein the agent is administered as a pharmaceutical comprising the agent and an acceptable pharmaceutical carrier.

21. A method of claim 20, wherein the agent is a GIP receptor antagonist.

22. A method of claim 21, wherein the GIP receptor antagonist comprises a 24 amino polypeptide corresponding to positions 7-30 of the sequence of GIP or effective alternatives thereto.

23. A method of claim 21, wherein the GIP receptor antagonist comprises at least an effective number of amino acids corresponding to positions 7-30 of the sequence of GIP or effective alternatives thereto.

24. A method of claim 20, wherein the pharmaceutical composition further includes an inert pharmaceutical excipient selected from the group consisting of sweetening, flavoring, coloring, dispersing, disintegrating, binding, granulating, suspending, wetting, preservative and demulcent excipients.

25. A method of claims 20, 21, 22 or 23, wherein the agent is lyophilized.

26. A method of claim 25, wherein the lyophilized agent is reconstituted with a suitable diluent selected from the group consisting of normal saline, sterile water, glacial acetic acid, sodium acetate and combinations thereof.

27. A method of preventing, inhibiting, treating or reducing nonalcoholic fatty liver disease in a human comprising:
instructing the human to take or a person to deliver to the human an effective amount of an agent to prevent and/or reverse hyperinsulinemia and/or insulin resistance to prevent, inhibit, treat or reduce nonalcoholic fatty liver disease in the human.

28. A method of claim 27, wherein the agent is a GIP receptor antagonist.

29. A method of claim 28, wherein the GIP receptor antagonist is selected from the group consisting of those set forth in Table I.

30. A method of claim 27, wherein the human takes the agent orally or by injection or the person delivers the agent orally, by injection or by gene therapy.

31. A method of claim 28, wherein the GIP receptor antagonist comprises a 24 amino polypeptide corresponding to positions 7-30 of the sequence of GIP or effective alternatives thereto.

32. A method of claim 28, wherein the GIP receptor antagonist comprises at least an effective number of amino acids corresponding to positions 7-30 of the sequence of GIP or effective alternatives thereto.

33. A method of preventing, inhibiting, treating or reducing nonalcoholic fatty liver disease in a human comprising:
instructing the human to take or a person to deliver to the human an effective amount of an agent to antagonize, block, inhibit or ablate the receptor to Glucose-Dependent Insulinotropic Polypeptide (GIP) to prevent, inhibit, treat or reduce nonalcoholic fatty liver disease in the human.

34. A method of claim 33, wherein the agent is a GIP receptor antagonist.

35. A method of claim 34, wherein the GIP receptor antagonist is selected from the group consisting of those set forth in Table I.

36. A method of claim 33, wherein the human takes the agent orally or by injection or the person delivers the agent orally, by injection or by gene therapy.

37. A method of claim 34, wherein the GIP receptor antagonist comprises a 24 amino polypeptide corresponding to positions 7-30 of the sequence of GIP or effective alternatives thereto.

38. A method of claim 34, wherein the GIP receptor antagonist comprises at least an effective number of amino acids corresponding to positions 7-30 of the sequence of GIP or effective alternatives thereto.

39. A method of claim 34, wherein an effective amount of the GIP receptor antagonist is an amount that is sufficient to inhibit GIP or GIP receptor activity in the range of from about 10% to about 100%.

40. A method of claim 34, wherein an effective amount of the GIP receptor antagonist is an amount that is sufficient to inhibit GIP or GIP receptor activity in the range of from about 40% to about 100%.

41. A method of claim 34, wherein an effective amount of the GIP receptor antagonist is an amount that is sufficient to inhibit GIP or GIP receptor activity in the range of from about 50% to about 100%.

42. A method of claim 34, wherein an effective amount of the GIP receptor antagonist is an amount that is sufficient to inhibit GIP or GIP receptor activity in the range of from about 40% to about 80%.

43. A method of claim 34, wherein an effective amount of the GIP receptor antagonist is an amount that is sufficient to inhibit GIP or GIP receptor activity in the range of from about 50% to about 80%.

44. A method of claim 34, wherein an effective amount of the GIP receptor antagonist is an amount that is sufficient to inhibit GIP or GIP receptor activity in the range of from about 40% to about 75%.

45. A method of claim 34, wherein an effective amount of the GIP receptor antagonist is an amount that is sufficient to inhibit GIP or GIP receptor activity in the range of from about 50% to about 75%.

46. A method of claim 34, wherein an effective amount of the GIP receptor antagonist is an amount that is sufficient to inhibit insulin release in the range of from about 10% to about 100%.

47. A method of claim 34, wherein an effective amount of the GIP receptor antagonist is an amount that is sufficient to inhibit insulin release in the range of from about 40% to about 100%.

48. A method of claim 34, wherein an effective amount of the GIP receptor antagonist is an amount that is sufficient to inhibit insulin release in the range of from about 50% to about 100%.

49. A method of claim 34, wherein an effective amount of the GIP receptor antagonist is an amount that is sufficient to inhibit insulin release in the range of from about 40% to about 80%.

50. A method of claim 34, wherein an effective amount of the GIP receptor antagonist is an amount that is sufficient to inhibit insulin release in the range of from about 50% to about 80%.

51. A method of claim 34, wherein an effective amount of the GIP receptor antagonist is an amount that is sufficient to inhibit insulin release in the range of from about 40% to about 75%.

52. A method of claim 34, wherein an effective amount of the GIP receptor antagonist is an amount that is sufficient to inhibit insulin release in the range of from about 50% to about 75%.

53. A method of claim 33, wherein the agent is an antisense molecule.

54. A method of preventing the development and/or reversing the process of NAFLD in an animal comprising:
administering and/or delivering to an animal an effective amount of a GIP-receptor antagonist in any form to prevent the development and/or reverse the process of NAFLD in the animal.

55. A method of claim 54, wherein the GIP receptor antagonist is selected from the group consisting of those set forth in Table I.

56. A method of claim 54 wherein the GIP-receptor antagonist is administered to the animal orally, by injection or by gene therapy.

57. A method of claim 54, wherein the GIP receptor antagonist comprises a 24 amino polypeptide corresponding to positions 7-30 of the sequence of GIP or effective alternatives thereto.

58. A method of claim 54, wherein the GIP receptor antagonist comprises at least an effective number of amino acids corresponding to positions 7-30 of the sequence of GIP or effective alternatives thereto.

59. A method of claim 54, wherein the GIP receptor antagonist is administered as a pharmaceutical comprising the agent and an acceptable pharmaceutical carrier.

60. A method of preventing the development and/or reversing the process of NAFLD in a human comprising:
instructing the human to take or a person to deliver to administer to the human an effective amount of a GIP-receptor antagonists in any form to prevent the development and/or reverse the process of NAFLD in the human.

61. A method of claim 60, wherein the GIP receptor antagonist is selected from the group consisting of those set forth in Table I.

62. A method of claim 60 wherein the GIP-receptor antagonist is administered to the animal orally, by injection or by gene therapy.

63. A method of claim 60, wherein the GIP receptor antagonist comprises a 24 amino polypeptide corresponding to positions 7-30 of the sequence of GIP or effective alternatives thereto.

64. A method of claim 60, wherein the GIP receptor antagonist comprises at least an effective number of amino acids corresponding to positions 7-30 of the sequence of GIP or effective alternatives thereto.

65. A method of claim 60, wherein the GIP receptor antagonist is administered as a pharmaceutical comprising the agent and an acceptable pharmaceutical carrier.

66. A method of claim 60, wherein the GIP receptor antagonist is an antisense molecule.
